(19) United States
(12) Patent Application Publication
(10) Pub. No.: US 2006/0019303 A1
(43) Pub. Date: Jan. 26, 2006

(54) Method to identify and analyze genes having modified expression in stimulated T cells

(75) Inventors: Brian Ervin Castle, Monroe, CT (US); Jianfei Yang, Sandy Hook, CT (US); Xiang Li, New Milford, CT (US); Jun Li, Danbury, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim Pharmaceuticals, Inc., Ridgefield, CT (US)

(21) Appl. No.: 11/187,572

(22) Filed: Jul. 22, 2005

Related U.S. Application Data
(60) Provisional application No. 60/590,733, filed on Jul. 23, 2004.

(51) Int. Cl. C12Q 1/68

(52) U.S. Cl. 435/6

(57) ABSTRACT

A method of identifying genes involved in the stimulation of primary T cells comprising the steps of: a) contacting experimental cells with a stimulating agent; b) preparing RNA from said experimental cells at one or more stimulation phases; c) measuring the level of gene expression in the cells; d) comparing the levels of gene expression of said experimental cells to the level of gene expression in control cells that have not been exposed to an stimulation agent; e) identifying genes that are up regulated or down regulated in said experimental cells relative to said control cells.

### RELATED APPLICATIONS

This application claims benefit to U.S. provisional application No. 60/590,733 filed on Jul. 23, 2004 and the contents of which are incorporated herein.

### FIELD OF THE INVENTION

The invention relates to the field of cell biology and inflammatory diseases and in particular to methods for identification and analysis of genes having modified expression in activated CD⁺4 cells.

### BACKGROUND INFORMATION

The differentiation of naive CD4⁺ T cells into subsets of T helper cells is a pivotal process with significant implications for host defense and the pathogenesis of immune-mediated diseases. Upon antigen exposure through contact with cells of the innate immune system, naive T cells undergo rapid clonal expansion and differentiation. The maturation of naive CD4⁺ T lymphocytes into full effector cells is a complex process comprising differential gene expression of cytokines, transcription factors and signaling molecules. These genes play important functions in T cell development and Tₕ1/Tₕ2 cytokine production.

There have been studies on genes induced upon CD4⁺ cell stimulation that have employed methods for single gene analysis. Individual genes such as EBI3, which functions in Tₕ1 cytokine production, have been identified as induced genes in activated naïve T cells (Pflanz et al. Immunity 2002; 16: 779-790; Chen et al. Nature 2001; 407: 916-920). There is a publication on gene expression profiling analysis during differentiation of CD8⁺ cells (J Biol Chem. 2003, 278:17044-52).

Microarray is a technique used to analyze the expression of a large number of genes simultaneously (see Debouck et al., (July 2002) Genetics 21: 48-50 and Current Protocols in Molecular Biology, John Wiley and Sons, July 2002). Microarray analysis can be performed in a number of different ways. Microarray analysis can be performed with DNA microarrays which contain microscopic spots of about 1 kb DNA sequences representing thousands of genes bound to the surface of glass microscopic slides. Microarray analysis can also be performed with oligonucleotide arrays (DNA chips) or high density nucleotide probes which contain synthetic oligonucleotides representing thousands of gene sequences synthesized on the surface of small areas of a glass slide.

Microarrays can be used to study the expression profiles of cells and tissues of significance in the study of a variety of diseases (Debouck et al., Annu. Rev. Pharmacol. Toxicol. 2000, 40: 193-208). Microarray techniques have been used to study the expression profile of T cells during the process of thymocyte selection (Schmitz et al, 2003, Int Immunol 15:1237-1248) or to identify genes that are regulated by TGF-β during early T cell polarization (Lund et al, J Immunol, 2003, 171:5328-5336). These approaches however did not address questions such as which genes are induced or repressed in naïve CD4+ T cells (CD4⁺CD8⁻CD45RO⁻) by TCR stimulation.

### BRIEF SUMMARY OF THE INVENTION

The present invention provides to a method for the analysis and identification of the level of expression profiles of thousands of CD4⁺ expressed genes simultaneously. Genes that are identified as having modified expression in stimulated T cells by the method of the invention could be considered as candidate genes as drug discovery targets in the field of autoimmune and inflammation.

The present invention provides a method of identifying and analyzing genes having modified expression in naïve peripheral blood T cells comprising the steps of:
a) contacting experimental cells with a stimulating agent;
b) preparing RNA from said experimental cells at one or more stimulation phases;
c) measuring the level of gene expression in the cells;
d) comparing the levels of gene expression of said experimental cells to the level of gene expression in control cells that have not been contacted to a stimulating agent;
e) identifying genes that are up regulated or down regulated in said experimental cells relative to said control cells.

Preferred embodiments of the invention include use of the method with cells chosen from the following cells types CD4+, CD8⁻ and CD45RO⁻⁻, CD4⁺/CD8⁺/CD45RO⁺, CD4⁺CD25⁺, CD4⁺CD25 cells.

In another preferred embodiment of the invention the stimulating agent is selected from antibodies directed against CD3, CD28, CD3 and CD28.

In another embodiment of the invention the experimental cells are stimulated with a combination of stimulating agents such as antibodies directed against CD3, CD28, CD3 and CD28, PMA, PMA+Ionomycin, or PMA and CD3.

In another embodiment, RNA samples are measured at the early, and late stages of CD4⁺ cells after stimulation.

In another embodiment the early phase is from between 5 minutes and 4 hours after administration of a stimulation agent. In another embodiment early phase is from between 30 minutes to 3 hours after administration of a stimulation agent. In another embodiment the late stage is from between 16 and 48 hours after administration of the stimulating agent. In another embodiment the late stage is from 12 to 24 hours after administration of the stimulating agent.

The method of the invention can be used for identifying and analyzing genes that may be targets for the development of inhibitor compounds useful in the treatment of, inflammatory, allergic and autoimmune diseases. Such diseases may be treated through the administration of a pharmaceutically acceptable amount of a compound that can inhibit the activity of genes identified and analyzed using the methodology explained herein.

In another embodiment of the invention gene expression is measured using microarray analysis.

The invention also provides a method for T cell depletion therapy that employs conjugated antibodies that are directed to antigens of cell surface proteins whose expression is modified in stimulated T cells as identified herein as well as genes identified using the disclosed methods.

### BRIEF DESCRIPTION OF THE DRAWINGS

FIG. 1 shows the gene expression data of selected genes in cells which are stimulated by antibodies directed to CD3 and/or CD28 in naïve CD4⁺ cells after 2 hours of stimulation. It is understood that antibodies directed to CD3 and/or CD28 can be used to stimulate CD4⁺ cells. These antibodies may be referred to herein as anti CD3 or α-CD3 and anti CD28 and αCD28. Fold change values were derived from the comparison of anti CD3 and anti CD28 stimulated vs. unstimulated CD4+ T cells. Fold change values derived from the comparison of anti CD3 alone (or anti CD28 alone) stimulated vs. unstimulated cells are also listed.

FIG. 2 shows the expression data of selected genes which are repressed in anti CD3 and anti CD28 stimulated naïve CD4⁺ cells at 24 hours. Fold change values were derived from the comparison of anti CD3 and anti CD28 stimulated vs. unstimulated CD4+ T cells. Fold change values derived from the comparison of anti CD3 alone (or anti CD28 alone) stimulated vs. unstimulated cells are also listed.

FIG. 3 shows the expression data of selected genes which are activated in anti CD3 and anti CD28 stimulated naïve CD4⁺ cells at 2 hours. Fold change values were derived from the comparison of anti CD3+ anti CD28 stimulated vs. unstimulated CD4+ T cells. Fold change values derived from the comparison of anti CD3 alone (or anti CD28 alone) stimulated vs. unstimulated cells are also listed.

FIG. 4 shows the gene expression data of selected genes which are repressed in anti α-CD3+ and anti CD28 stimulated naïve CD4⁺ cells at 2 hours. Fold change values were derived from the comparison of anti CD3 and anti CD28 stimulated vs. unstimulated CD4+ T cells. Fold change values derived from the comparison of anti CD3 alone (or anti α-CD28 alone) stimulated vs. unstimulated cells are also listed.

### DETAILED DESCRIPTION OF THE INVENTION

Unless defined otherwise, the scientific and technological terms and nomenclature used herein have the same meaning as commonly understood by a person of ordinary skill in the art to which this invention pertains. The procedures for cell culture and general molecular biology methods and the like are common methods used in the art (see for example, Current Protocols in Molecular Biology, John Wiley and Sons, July 2002).
Tissue—refers to one or more cells, extracts and fractions thereof.
Cell—refers to cells in any form, including but not limited to, cells retained in tissue, cell clusters and individually isolated cells.
Naïve CD4⁺ cell—refers to cells of immunological origin that can be found resident in blood, spleen, and thymus for example. Upon antigen exposure through contact with cells of the innate immune system, naive T cells undergo rapid clonal expansion and differentiation. The maturation of naive CD4⁺ T lymphocytes into full effector cells is a process comprising differential gene expression of cytokines, transcription factors and signaling molecules. These genes play important functions in T cell development and Tₕ1/Tₕ2 cytokine production.
Gene transcription refers to a process whereby one strand of a DNA molecule is used as a template for synthesis of a complementary RNA by RNA polymerase.
Gene expression refers to the process whereby information encoded in a particular gene is decoded into a particular protein. The level of gene expression as the term is used herein can be can be determined by measuring the level of mRNA in a cell.
DNA refers to polynucleotide molecules, segments or sequences and is used herein to refer to a chain of nucleotides, each containing the sugar deoxyribose and one of the four adenine (A), guanine (G) thymine (T) or cytosine (C).
RNA refers to polynucleotide molecules, segments or sequences and is used herein to refer to a chain of nucleotides each containing the sugar ribose and one of the four adenine (A), guanine (G) uracil (U) or cytosine (C).
Oligo means a short sequence of DNA or RNA and their derivatives typically 8 to 35 nucleotides in length. The exact size of the molecule will depend on many factors, which in turn depend on the ultimate function or use of the oligonucleotide. An oligonucleotide can be derived synthetically, by cloning or by amplification. The term “derivative” is intended to include any of the above described variants when comprising an additional chemical moiety not normally a part of these molecules. These chemical moieties can have varying purposes including, improving a molecule's solubility, absorption, biological half life, decreasing toxicity and eliminating or decreasing undesirable side effects.
Autoimmune and inflammatory disease as used herein means diseases that are associated with autoimmune and inflammatory conditions such as inflammatory and autoimmune conditions such as osteoarthritis, reperfusion injury, asthma, multiple sclerosis, Guillain-Barre syndrome, Crohn's disease, ulcerative colitis, psoriasis, graft versus host disease, systemic lupus erythematosus, rheumatoid arthritis, Alzheimer's disease, toxic shock syndrome, insulin-dependent diabetes mellitis, acute and chronic pain as well as symptoms of inflammation and cardiovascular disease, stroke, myocardial infarction alone or following thrombolytic therapy, thermal injury, adult respiratory distress syndrome (ARDS), multiple organ injury secondary to trauma, acute glomerulonephritis, dermatoses with acute inflammatory components, acute purulent meningitis or other central nervous system disorders, Grave's disease, myasthenia gravis, scleroderma and atopic dermatitis.
Cell Line—refers to cells capable of stable growth in vitro for multiple generations.
Stimulation—also referred to herein as “activation” refers to the process in CD4⁺ cells whereby upon presentation of an antigen through contact with cells of the innate immune system, naive T cells undergo rapid clonal expansion and differentiation.
Stimulating agent—The term “stimulating agent” includes any chemical, physical, biological, electrical or radiation treatment, stimulus or condition which is capable of causing stimulation of CD4⁺ cells. It is understood that different stimulating agents may be used depending on the type of T cell used. The selection and use of suitable stimulating agents are known to those skilled in the art. Preferred stimulating agents include anti CD3, anti CD28, and combinations thereof.
Stimulation phase—the term “stimulation phase” refers to a particular stage of the T cell stimulation. A stimulation phase may correspond to a distinct cellular or metabolic event such as the onset of gene expression of a subset of genes. The term stimulation phase as used herein is also understood to be descriptive of a temporal stage of stimulation (i.e. early or late phases). In the case of T cell stimulation the initial stimulation phases can be immediately early and late stages of stimulation. The late stage corresponds to between about 16 and 72 hours after administration of a stimulating agent and is generally associated with the expression of STAT1, EBI3, IFN-γ, GM-CSF Genes that are modulated at the late stage are linked to cytokine biosynthesis and CD4+ cell homeostasis. Some genes such as interleukin 3 are induced at 2 hours but more expression is observed at 24 hours.
Target—refers to any gene perturbed in a disease state, developmental stage or drug treatment. Frequently a target refers to a drug development target that is capable of being altered by an agent or compound. Such drug development targets are suitable for screening candidate compounds in direct binding assays.
Hybridization—Association of two complementary nucleic acid strands or analogues thereof to form a double stranded molecule which can contain two DNA strands, two RNA strands, or one DNA strand and one RNA strand.
Up regulated—refers generally to an increase in level of gene expression normally in response to a stimulation agent as herein defined. The expression of a gene is considered up regulated if the level of expression is at least 120 percent relative to control, preferably 150 percent relative to control and most preferably 200 percent or higher relative to control.
Down regulated—refers generally to a decrease in the level of gene expression normally in response to a stimulation agent as herein defined. The expression of a gene is considered down regulated if the level of expression is less than 80 percent relative to control, preferably less than 60 percent relative to control and most preferably 50 percent or lower relative to control.
DNA Microarray—refers collectively to a technique(s) used to measure and analyze the expression of a large number of genes simultaneously and as described in Microarray Analysis, Schena, Mark Wiley-Liss, 2003 incorporated herein by reference. The term can refer to DNA microarrays which contain microscopic spots of about 1 kb DNA sequences representing thousands of genes bound to the surface of glass microscopic slides. The term can also refer to oligonucleotide arrays (DNA chips) or high density nucleotide probes which contain synthetic oligonucleotides representing thousands of gene sequences synthesized on the surface of small areas of a glass slide.

The method of the invention provides a general approach to study stimulation in primary T cells such as naïve CD4⁺ cells. The preferred method of the invention uses CD4⁺ cells. The invention provides a method to identify and analyze genes that are modified (i.e. up-regulated or down-regulated) at different stimulation stages in T cells. Using the method of the invention, many aspects of the stimulation of T cells can be studied in a single experiment/method. The method of the invention provides data on gene expression at one or more stimulation phase. Data obtained through the use of the method can provide a rationale to prioritize genes as candidates for target validation in the field of inflammation and autoimmune disease. Thus, genes identified using the method of the invention and that are disclosed herein are useful as biomarkers for stimulated T cells.

The invention also provides methods for therapy of autoimmune and inflammatory conditions. Genes that encode cell surface proteins that are upregulated in stimulated T cells serve as suitable candidates for use in T cell depletion therapy.

A large number of newly identified genes in the human genome show no significant sequence similarity to genes with known function. Therefore, these genes are not easily recognized as drug targets. Expression analysis is an alternative method to suggest a possible function for a given gene. (Mini Rev. Med. Chem. (2001) 1:197-205). The link between the modulation of gene expression resulting in phenotypic or functional changes is well established. In CD4⁺ cells for example, IL-2, CD154 (CD40 Ligand), IL-2Rα, IL-4, IL-5 GM-CSF, TNF-α, and IFN-γ are produced after stimulation through the T cell receptor. The mRNA levels for the early response change within 2 h following stimulation. This increase in mRNA levels are typically followed by increased protein levels and increased activity of this protein which may be measured by cytokine ELISA assays and/or western blots.

Thus, novel methods aimed at studying temporal gene expression in CD4+ cells as described herein are likely to lead to the identification of genes with functional relevance in CD4+ cell physiology. Many of these functionally important genes will play a role in immunological and inflammatory responses.

The present invention also provides a method for finding novel genes and/or novel functions for known genes. In the case of naïve CD4⁺ stimulated cells of particular interest are genes having one or several of the following characteristics including but not limited to i) involvement in TCR response ii) CD4+ T cell specific iii) involvement in Th1 cytokine production iv) involvement in Th2 cytokine production v) involvement in IL-2 production and vi) involvement in T cell homeostasis (proliferation and apoptosis). Transcription factors could mediate the differentiation of naïve CD4+ T cells to Th1 or Th2 cells. The secreted or cell-surface genes could also function as pro-inflammatory mediators (adhesion, chemotaxis, growth factor) for other cell types. The method of the invention can be used to identify genes having the properties listed above.

In the method of the invention, the level of gene expression in cells exposed to an stimulating agent is compared to the level of gene expression of control cells that have not been treated with the stimulating agent (control) at one or more stimulation phases. In the case of naïve CD4+ cells, the level of gene expression is measured at stimulation phases. It is also contemplated that more than one measurement of gene expression levels can be determined in an individual stimulation phase. A stimulation phase is measured on the amount of time elapsed after administration of a stimulating agent to the experimental cells. Although samples can be collected at any time during or after naive CD4+ cell stimulation, samples are preferably collected at about 2 hours and 24 hours. Genes that show significant induction or reduction at the 2 h time point are mostly likely mediated directly through the TCR signaling. Genes that are up-regulated or down-regulated at the 24 h time point include genes that are directed regulated by the TCR signaling or indirectly regulated by the TCR signaling pathways. Data on the level of gene expression at varying time points after T cell stimulation can provide information about the function of the genes as discussed herein.

The source of the cells can be tissues, tissue culture cells or cell extracts. Material collected from any of the sources is collectively referred to as “cells.” Preferably, the cells are obtained from tissue and most preferably cells of CD4⁺ lineage such as peripheral blood. The cells are generally cultured for less than 1 hour in Iscove's Modified Dullbecco's medium before a stimulating agent is administered. In other embodiments of the invention the CD4⁺ cells can be obtained from different sources such as the spleen or tonsil.

The stimulating agent used can be a chemical, physical, biological, electrical or radiation treatment or a condition that is capable of producing a biological response such as CD4+ cell stimulation. The preferred stimulating agents are chemical and biological agents and CD4⁺ cell stimulation can be obtained through stimulation by a CD3+ α-CD28. Stimulating agents should be used in the manner and or amounts to promote the biological response. In the case of CD4⁺ cells, stimulating agents are used in such a manner as to promote CD4+ cell stimulation.

Levels of gene expression are determined from analysis of RNA isolated from cells and/or tissues after administration of an stimulating agent.

Methods of RNA isolation are well known in the art and the RNA isolation method used should depend on the source of the cells (see Maniatis et al., Molecular Cloning: A laboratory Manual, Third Edition (2001), Cold Spring Harbor Press, Cold Spring Harbor, N.Y.). The preferred method of RNA isolation is the Qiagen RNA purification kit. (Qiagen, Valencia, Calif.).

Steps should be taken to avoid degradation of the RNA prior to analysis. Typically, RNA is isolated from cells soon after the cells have been collected for analysis. Cells that have been collected should be stored under conditions that limit the degradation of RNA known to those skilled in the art. Likewise, after RNA has been isolated from the cell samples the RNA should be stored under conditions that reduce RNA degradation. For example, RNA should be stored on dry ice or at −70° C. under RNAse free conditions. DEPC water should be used in buffers and solutions. Conditions should also be maintained such that additional RNA synthesis is terminated when the cells are collected. In this way RNA expression will be representative of the types and levels of RNA expression at the time of collection.

Isolated RNA from the cells is used to synthesize double stranded DNA in a reverse transcriptase reaction that can be performed according to methods known to those skilled in the art. The preferred reverse transcriptase is the Superscript reverse transcriptase (Superscript Choice™, Invitrogen Carlsbad, Calif.). It is used according the manufacturers instructions. Approximately 5 to 15 μg total RNA from each time point is used in reverse transcriptase reactions, however, the amount of RNA used varies depending on the number of genes tested and the method used to detect gene expression as apparent to those skilled in the art.

The cDNA is used as a template for the synthesis of labeled cRNA with a plasmid or vector. The cRNA can be labeled with fluorescence or with other methods commonly used in the art such as for labeling nucleic acids. The cRNA is most preferably labeled with biotin. The cRNA is then fragmented using an alkaline base method commonly used in the art.

### Analysis of Gene Transcription

RNA levels can be measured using a number of techniques available to those skilled in the art. Quantitative methods for detecting specific RNA levels of certain genes can be used such as Northern hybridization, PCR analysis, or microarray analysis. The preferred method of RNA analysis is microarray analysis.

Laboratory materials and equipment for performing microarray analysis are available from companies such as Affymetrix, Agilent and Spotfire. Microarray or cRNA chip analysis offer the advantage of being able to analyze multiple genes in a single experiment. Preparation of cRNA and hybridization are performed according to methods commonly used in the art. Microarray analysis can be performed using procedures available from various companies such as Affymetrix and Spotfire.

The Affymetrix procedure is the preferred method. The samples can be hybridized to the human genome U133 microarray which is comprised of two microarrays of over 1,000,000 oligonucleotides covering more than 39,000 transcript variants representing 33,000 human genes. The samples can also be hybridized to the Rat genome U34 set which contains more than 24,000 known genes and EST clusters. The U34 array consists of U34A, U34B and U34C chips and can be performed essentially as follows: Between 5 and 15 μg of the total RNA can be converted into double stranded cDNA by reverse transcription using a cDNA synthesis kit. The preferred kit for cDNA synthesis is Superscript Choice™, Invitrogen (Carlsbad, Calif.) which has a special oligo (dT024 primer) (Genset, La Jolla, Calif.) containing a T7 RNA polymerase promoter site added 3′ of the poly T tract. After second strand synthesis, labeled cRNA is generated from the cDNA samples by an in vitro transcription reaction using a reporting reagent such as biotin-11-CTP and biotin-16-UTP (Enzo, Farmingdale, N.Y.). Labeled cRNA can be purified by techniques commonly used in the art. The preferred method is to use RNeasy™ spin columns (Qiagen, Valencia, Calif.). Current Protocols in Molecular Biology, John Wiley and Sons, July 2002. About 5 to 30 micrograms of each cRNA sample can be fragmented by mild alkaline treatment. Preferably, the cRNA sample is fragmented by treatment at 94° C. for 35 minutes in fragmentation buffer as suggested by the manufacturer. A mixture of control cRNAs for bacterial and phage genes was included to serve as tools for comparing hybridization efficiency between arrays and for relative quantitation of measured transcript levels. Before hybridization, the cRNA samples can be heated at about 94° C. for 5 minutes, equilibrated at 45° C. for 5 minutes and clarified by centrifugation (14,000×g) at room temperature for 5 min. Aliquots of each cRNA sample are hybridized to arrays, or stored according the manufacturer's directions. The arrays (U133A) are then washed according to methods commonly used in the art. The preferred wash is with non-stringent (6×SSPE, 0.01% Tween-20, 0.005% antifoam) and stringent (100 mm MES, 0.1M NaCl, 0.01% Tween 20), stained with R— Phycoerythrin Streptavidin- (Molecular Probes, Eugene, Oreg.), washed again and scanned by an argon-ion laser scanner with the 560-nm long-pass filter (Molecular Dynamics; Affymetrix). Data analysis can be performed in order to determine if a gene expression level is increased or decreased or unchanged. Preferably, software such as MAS 4.0 or MAS 5.0 software (Affymetrix, Calif.) is used for data analysis.

A gene would be considered to have modified expression in activated cells if the expression profile of the gene indicates that it is either up regulated or down regulated as the terms are defined herein. It is understood that when measuring expression levels using microarray analysis that the level of expression is reproducibly above the noise levels obtained from measurement of gene expression with a microarray apparatus. The noise level can vary depending on variables (such as quality of cRNA probes, sensitivity of detection and quality of oligos on the chip) that effect noise level

For instance, a gene would be considered to have modified expression in CD4⁺ cell stimulation if the gene were up regulated during the early stimulation phase, typically measured at 2 h. A gene would be a preferred candidate for involvement in CD4⁺ cell stimulation if the expression levels returned to normal levels relative to control during the late phase of stimulation.

It is also contemplated that the method of the invention can be used with other stimulation agents such as neuropeptides, chemokines, cytokines, small molecule activators of CD4⁺ cell stimulation such as IL-12+IL-18.

### PREFERRED EMBODIMENT OF THE INVENTION

The following examples are provided to illustrate the invention, but not to limit its scope. Other variants of the invention will be readily apparent to one of ordinary skill in the art. The contents of all references, patents and published patent applications cited throughout this application, as well as the figures and sequence listing are hereby incorporated by reference.

### Isolation of Peripheral Blood Cells and Their Stimulation

Primary CD4+ T cells were isolated from human peripheral blood by using StemCell Technologies Human Naïve CD4+ T cell Enrichment Cocktail (Catalog # 14165) according to manufacturer's published methods. Cells were resuspended in Iscoves Media +10% Fetal Calf Serum at a concentration of 2×106 per mL.

The purified T cells were incubated for 2 or 24 hrs at 37° C. in the presence of either anti CD3, anti CD28 or anti CD3 and anti CD28 which were coated at 10 μg/mL each in PBS for 24 hr at 4° C. The incubation was stopped at time=0, 2, 6, and 24 h.

### Stimulation Essay

Quantitative RT-PCR (Taqman) analysis was performed to determine the level of naïve CD4⁺ cell stimulation. mRNA expression of IL-2 and IFN-γ was quantitated by Taqman™ analysis to confirm the activation of naïve CD4⁺ T cells by anti-CD3 or anti-CD3+anti-CD28 stimulation.

### Preparation of cRNA

RNA was isolated from the samples using the RNAeasy™ total RNA isolation kit from Qiagen as described by the manufacturer. The homogenization solution was added directly to the cell pellet and homogenates were processed as recommended by the manufacturer. Between 5-10 μg of the total RNA was converted into double stranded cDNA by reverse transcription using a cDNA synthesis kit (Superscript Choice, Invitrogen custom synthesis was performed according to the Affymeterix protocol.

Preparation of cRNA was performed according to the manufacturer's protocol (Affymetrix, Santa Clara, Calif.). After second strand synthesis, labeled cRNA was generated from the cDNA samples by an in vitro transcription reaction supplemented with biotin-11-CTP and biotin-16-UTP (Enzo, Farmingdale, N.Y.). The labeled cRNA was purified by using RNeasy™ spin columns (Qiagen, Valencia, Calif.). Fifteen micrograms of each cRNA sample was fragmented by mild alkaline treatment at 94° C. for 35 minutes in fragmentation buffer (40 mM Tris-acetate, pH 8.1, 100 mM potassium acetate, 30 mM magnesium acetate) and then used to prepare 0.3 ml of master hybridization mix (100 mM MES, 1M [NaCl], 20 mm EDTA, 0.01% Tween 20, 0.1 mg/ml herring sperm DNA (Promega, Madison, Wis.), 0.5 mg/ml acetylated BSA (Invitrogen)). A mixture of control cRNAs, available from the manufacturer, for bacterial and phage genes was included in the mix (BioB, BioC, BioD, and cre, at 1.5, 5, 25 and 100 pM, respectively) to serve as tools for comparing hybridization efficiency between arrays and for relative quantitation of measured transcript levels. Before hybridization, the cRNA samples were heated at 94° C. for 5 minutes, equilibrated at 45° C. for 5 minutes and clarified by centrifugation (14,000×g) at room temperature for 5 min.

### Data Analysis

Data analysis was performed by using Affymetrix MAS5.0 software. The software includes algorithms that determine whether a gene is absent or present (detection call) and whether the expression level of a gene in an experimental sample is significantly increased or decreased (change call) relative to a control sample. To assess differences in gene expression, genes were selected based on fold change at 2 fold or more in conjunction with absolute call and difference call. Specifically, the following criteria were selected for significant changes for primary screen of each time point: (1) the change in the average difference across all probe sets was >2 fold; (2) for induced genes, a change call of “increase” or “marginal increase” should be associated with the experimental sample; (3) for suppressed genes, a change call of “decrease” or “marginal decrease” should be associated with the control sample.

### T Cell Depletion Therapy

The invention also provides methods for therapy of autoimmune and inflammatory immune conditions. Genes that encode cell surface proteins that are upregulated in stimulated T cells serve as suitable protein target candidates for use in T cell depletion therapy. One example where T cell depletion therapy can be useful is in the case of host graft disease. Elimination of T cells from a bone marrow graft from a tissue donor may reduce the chance of an immune reaction against the recipient's tissues. In one embodiment of the invention T cell therapy can be performed by the steps of administration to a patient in need thereof of a therapeutically effective amount of an antibody specifically directed to an antigen of a cell surface protein that is upregulated in stimulated T cells wherein said antibody is conjugated to a cytotoxin capable of killing or otherwise depleting a T cell. Thus, the antibody when administered will bind to target protein being expressed on the T cell surface releasing the effect of the cytotoxin conjugated to the antibody and killing the T cell. The target cell surface protein can be selected from the group of proteins upregulated in stimulated T cells disclosed herein or an upregulated protein identified using the methods for identifying genes upregulated in T cells identified herein. Cell surface proteins can be distinguished from non cell surface proteins using methods known in the art such as there presence of domains characteristic of cell surface proteins. Cell surface proteins can be identified based on the existence of signal peptide for secretion, transmembrane domain(s) and sequence/structure similarity to known cell surface proteins. Preferred cell surface proteins include the following: tumor necrosis factor receptor superfamily, member 5, CD38 antigen, Epstein-Barr virus induced gene 3, 19A24 protein, interleukin 15 receptor, alpha, sodium channel, voltage-gated, type I, alpha polypeptide, dystrobrevin, alpha, antigen identified by monoclonal antibody MRC OX-2, FOS-like antigen 1, CD69 antigen, CED-6 protein). Methods for conducting T cell depletion by antibody-dependent cellular cytotoxicity (ADCC) are known in the art and are disclosed in (Cancer Res. 2004 64:2127-2133;) the contents of which are incorporated herein.

### RESULTS AND DISCUSSION

Using the method of the invention, it was found that the expression of many genes was increased or decreased after CD4+ cell stimulation as shown in Table 1, suggesting a role for these genes in T cell stimulation.

TABLE I
Numbers of gene hits (2-fold or more)

| | | Overlap with | Down- | |
|---|---|---|---|---|
| | | Genes Up- | regulated | Overlap with |
| | Up-regulated | regulated by | by anti | Genes Down- |
| Stimulation | by anti | anti CD3 | CD3 + | regulated by |
| time | CD3 + CD28 | alone | αCD28 | anti CD3 alone |
| 2 h | 423 | 288 | 365 | 101 |
| 24 h | 715 | 339 | 1179 | 159 |

In order to identify genes essential for CD4+ cell physiology the temporal peak for transcriptional activation and repression following CD4+ cell stimulation were determined.

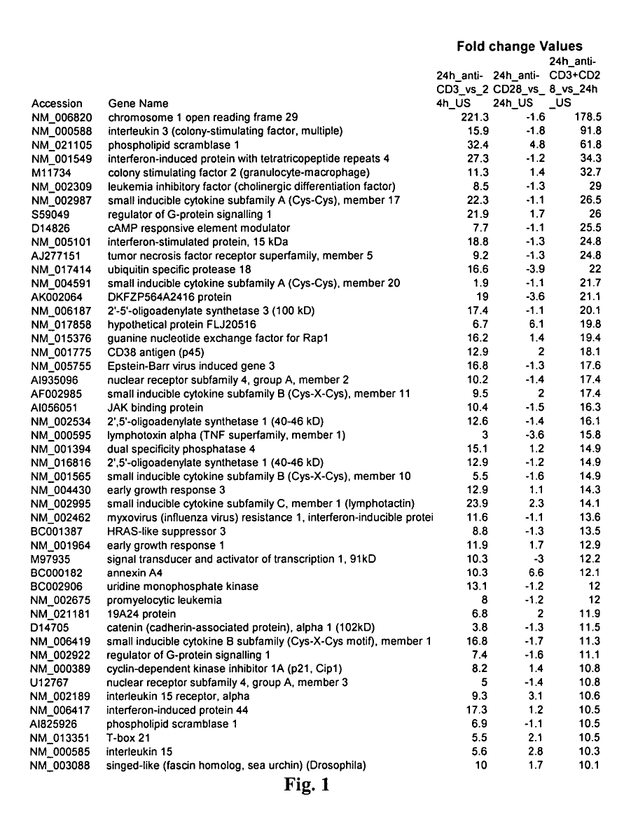

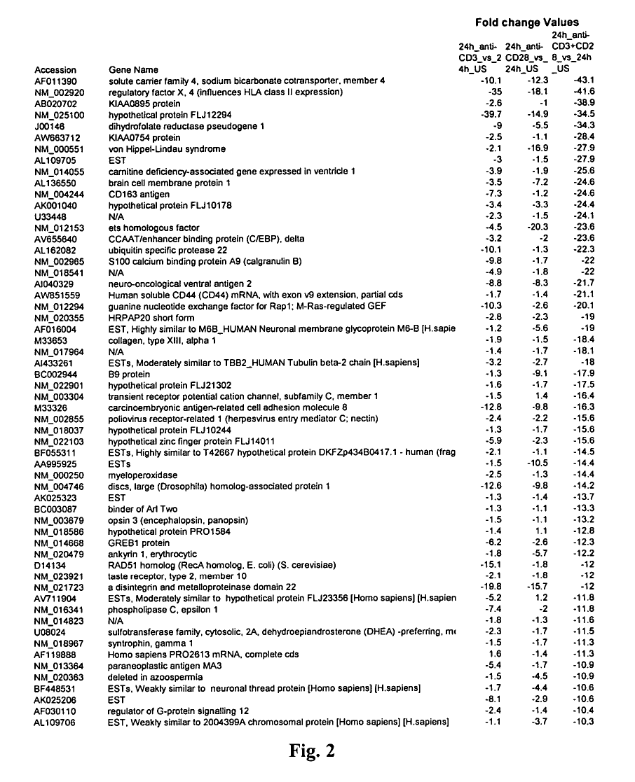

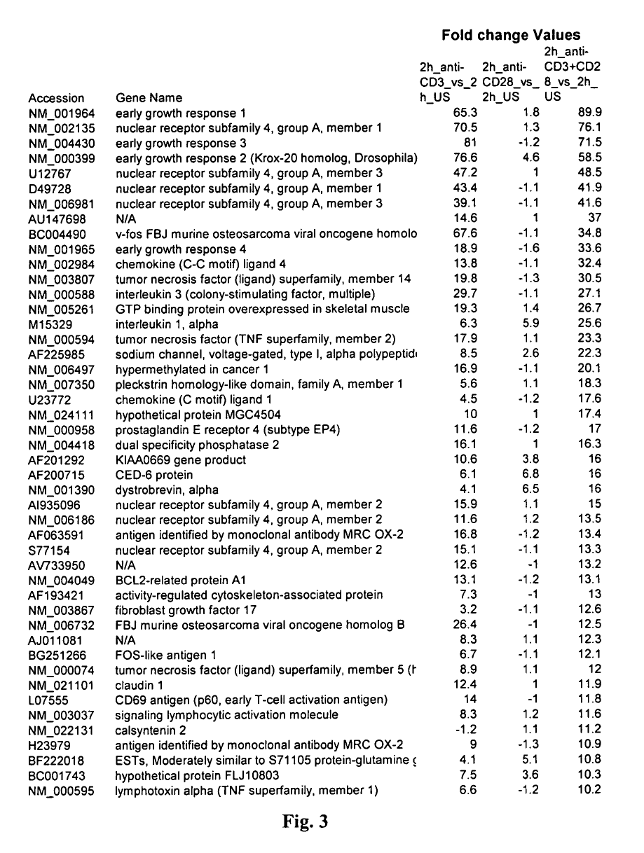

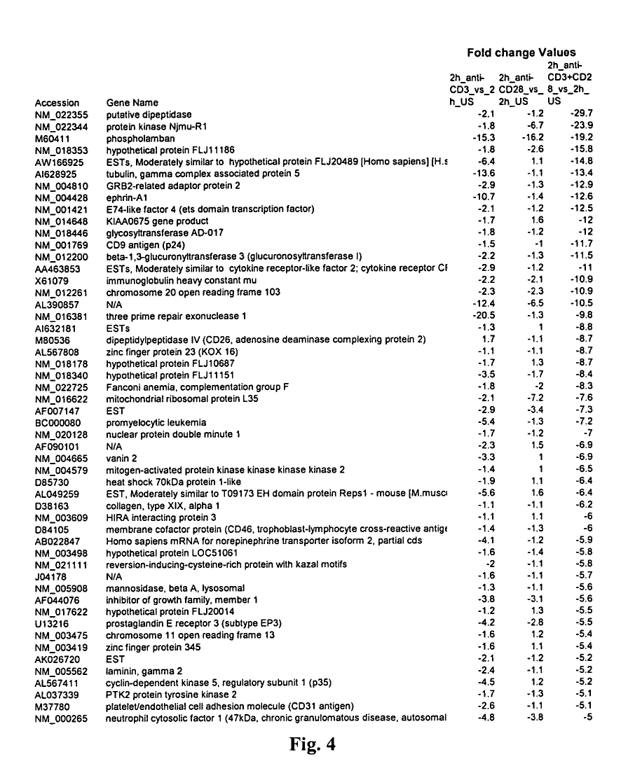

Our method allows for the temporal analysis of CD4+ cell gene expression profiles following stimulation. FIGS. 1-4 show representative genes that are up-regulated or down-regulated by anti CD3 and anti CD28 at 2 h or 24 h by ten fold or more. FIG. 4 shows elevation 5 fold or more. Each line represents the relative expression profile (in percentage) of a gene with 2-fold or more induction compared to the unstimulated control (see methods). The data shows the relative expression profile of a gene compared to a unstimulated control. Negative numbers mean regulated and positive numbers mean upregulated. We also performed expression analysis with CD4+ T cells that were stimulated with CD3 alone or CD28 alone. Both the anti CD3 stimulation and anti CD28 stimulation are required for CD4+ T cell proliferation in vivo. For gene expression at global level, genes that are significantly up-regulated by anti CD3 and anti CD28 are also induced by anti CD3 a-CD3 (FIG. 1 and FIG. 3).

### Genes Regulated at 2 h

Genes showing peak up regulation relative to T(0) at 2 h or less are considered early stimulation genes. FIG. 3 shows the fold change values of the genes that are induced >10 fold by anti CD3 and anti CD3 α-CD28 at 2 h. The gene profiling was done using the Spotfire software. Cytokines or chemokines such as TNF-α, IL-1α, IL-3, Lymphotoxin α, CCL-1, CCL-4 are induced >10 fold. Induced transcription factors and nuclear receptors include early growth response 1, early growth response 2, early growth response 3, early growth response 4, nuclear receptor subfamily 4, group A, member 1, nuclear receptor 25 subfamily 4, group A, member 2, nuclear receptor subfamily 4, group A, member 3. Many of these genes have not been reported to be associated with T cell activation. There are also less characterized full-length genes (such as KIAA0669 gene product and hypothetical protein FLJ10803) and ESTs such as EST-BF222018 and EST-AV733950). Early signal-dependent gene repression was also observed (FIG. 4), including transcription factors (E74-like factor 4, zinc finger protein 23) and enzymes (putative dipeptidase, protein kinase Njmu-R1, glycosyltransferase AD-017, dipeptidylpeptidase IV (CD26) and MAP4K2).

### Genes Regulated at 24 h

An important aspect of naïve CD4⁺ T cell physiology is that after stimulation, naïve CD4⁺ T cells produce cytokines and transcription factors which drive the differentiation of naïve CD4⁺ T cells into Th1 or Th2 effector cells. Genes that are up or down regulated after 24 hours of stimulation likely contribute to putative function of these genes in driving the differentiation of naïve CD4⁺ T cells. STAT1, a transcription factor that is known to promote the differentiation to Th1 cells, is induced >10 fold by anti CD3 and anti CD3 and anti CD3 αCD28 or by anti αCD3 alone (FIG. 1). In addition, EBI3 or IL-27, which was induced 17 fold at 24 h of stimulation (FIG. 1), was a recently identified cytokine which synergizes with IL-12 to trigger IFN-γ production of naïve CD4⁺ T cells (Pflanz, S. et al., Immunity, 2002, 16:779-90). Both IL-15 and IL-15 receptor alpha are induced >10 fold at 24 hours of stimulation (FIG. 1). Naïve T cells are dependent on cytokines for survival, especially γC-family cytokines such as IL-15. CD8⁺ memory T cells are also highly dependent on IL-15 (Zhang, X. et al, 1998, Immunity, 8:591-599). Although the expression of IL-15Rα is known to be induced in activated T cells (Schluns, K. S. et al., 2003, Nat Rev Immun, 3:269-279), it has not been previously known that IL-15 is highly induced naïve CD4 cells by TCR stimulation. Several genes that are induced at 2 h remain at high expression after 24 h of stimulation suggesting that they play important roles in maintaining the functions of activated CD4⁺ T cells. These genes include IL-3, lymphotoxin α, tumor necrosis factor receptor superfamily, member 5, early growth response 1, early growth response 3, early growth response 4, nuclear receptor subfamily 4, group A, member 1, nuclear receptor subfamily 4, group A, member 3, nuclear receptor subfamily 4. We observed up-regulation of genes in the ISG15-mediated post-translational modification pathway. Both ISG-15 and the ISG15-specific protease USP18, are induced >20 fold at 24 h of anti CD3+ anti CD28 stimulation. ISG15 is a ubiquitin like molecule and can be conjugated to signaling molecules such as STAT1 to modify their functions (Kim, K. I. et al., 2003, Biochem Biophys Res Commun, 307:431-434). The novel observation of this pathway linking to T cell activation provides new opportunities for targeting T cell-mediated immunological diseases.

1. A method of identifying and analyzing genes having modified expression in stimulated primary T cells comprising the steps of:
a) contacting experimental cells with a stimulating agent;
b) preparing RNA from said experimental cells at one or more stimulation phase;
c) measuring the level of gene expression in the cells;
d) comparing the levels of gene expression of said experimental cells to the level of gene expression in control cells that have not been contacted with a stimulation agent;
e) identifying genes that are up regulated or down regulated in said experimental cells relative to said control cells.

2. The method of claim 1 using CD4+ Cells selected from the list consisting of CD4⁺ cells, CD8⁻, CD45RO⁻, CD4⁺/CD8⁺/CD45RO⁺, CD4⁺CD25⁺ and CD4⁺CD25⁻.

3. The method of claim 1 wherein the stimulation phases are selected from early and late stage stimulated CD4⁺ cells.

4. The method of claim 3 wherein the early phase is from between 5 minutes and 4 hours after administration of a stimulation agent.

5. The method of claim 4 wherein the early phase is from between 30 minutes to 3 hours after administration of a stimulation agent.

6. The method of claim 3 wherein the late stage is from between 16 and 48 hours after administration of the stimulating agent.

7. The method of claim 6 wherein the late stage is from 12 to 24 hours after administration of the stimulating agent.

8. The method of claim 1 wherein the experimental cells are stimulated with a stimulating agent selected from the list consisting of antibodies directed to CD3, CD28, PMA, PHA, ionomycin, ICAM, IL-12 and IL-18.

9. The method of claim 8 wherein said stimulating agent is selected from the list consisting of antibodies directed to CD3, CD28.

10. The method of claim 1 wherein the gene is identified as up regulated when the level of gene expression is elevated at least 120 percent relative to control.

11. The method of claim 10 wherein the gene is identified up regulated when the level of gene expression is elevated at least 200 percent relative to control.

12. The method of claim 1 wherein the gene is identified as down regulated when the level of gene expression is less than 80 percent relative to control.

13. The method of claim 1 wherein the gene is identified as down regulated if the level of gene expression is less than 50 percent relative to control.

14. The method of claim 1 wherein levels of a gene expression are measured using microarray analysis.

15. A method of treating chronic inflammation in humans said method comprised of the step of administering to a human in need thereof a pharmaceutically acceptable amount of an inhibitor of a gene that is identified using the method of claim 1.

16. A method of treating chronic inflammation in humans said method comprised of the step of administering to a human in need thereof a pharmaceutically acceptable amount of an inhibitor of a gene that is upregulated in stimulated T cells according to FIG. 1 at either 2 hours or 24 hours.

17. A method of treating autoimmune or inflammatory disease by depleting T cells in a human said method comprised of the step of administering to a patient in need thereof a therapeutically effective amount of an antibody specifically directed to an antigen of a cell surface protein that is upregulated in stimulated T cells using the method of claim 1 wherein said antibody is conjugated to a cytotoxin capable of killing a T cell.

18. The method of claim 17 for the treatment of graft vs. host disease.

19. A method of depleting T cells in a human said method comprised of the step of administering to a patient in need thereof a thereapuetically effective amount of an antibody specifically directed to an antigen of a cell surface protein selected from the list consisting of: tumor necrosis factor receptor superfamily, member 5, CD38 antigen, Epstein-Barr virus induced gene 3, 19A24 protein, interleukin 15 receptor, alpha, sodium channel, voltage-gated, type I, alpha polypeptide, dystrobrevin, alpha, antigen identified by monoclonal antibody MRC OX-2, FOS-like antigen 1, CD69 antigen, or CED-6 protein).
